(19) United States
(12) Patent Application Publication
(10) Pub. No.: US 2003/0180349 A1
(43) Pub. Date: Sep. 25, 2003

(54) Respiratory infection prevention and treatment with terpene-containing compositions

(76) Inventor: Lanny U. Franklin, Atlanta, GA (US)

Correspondence Address: NEEDLE & ROSENBERG, P.C., The Candler Building, 127 Peachtree Street, N.E., Atlanta, GA, 30303-1811, US

(21) Appl. No.: 10/314,613

(22) Filed: Dec. 9, 2002

Related U.S. Application Data
(60) Provisional application No. 60/336,628, filed on Dec. 7, 2001.

(51) Int. Cl. A61K 9/127; A61K 31/125; A61K 31/045; A61K 35/78; A61K 31/015; A61K 31/12

(52) U.S. Cl. 424/450; 514/729; 514/739; 514/763; 514/690; 514/692; 424/742

(57) ABSTRACT

Composition and methods for prevention and treatment of a respiratory infection. A composition comprising a single terpene, a terpene mixture, or a liposome-terpene(s) composition is disclosed. The composition can be a true solution of an effective amount of an effective terpene and a carrier such as water. The composition can be a suspension or emulsion of terpene, surfactant, and carrier. The composition(s) of the invention can be administered before or after the onset of the disease. Administration can be, for example, by spraying the respiratory tract with a solution of the present invention. Prevention and treatment of a respiratory infection by the inhalation of a solution containing a single bioactive terpene, a bioactive terpene mixture, or a liposome-terpene(s) composition before or after the onset of the infection is described. A true solution of terpene and water can be formed by mixing terpene and water at a solution-forming shear rate in the absence of a surfactant.

### CROSS REFERENCE TO RELATED APPLICATIONS

This application claims the benefit of U.S. Provisional Application No. 60/336,628, filed Dec. 7, 2001.

### BACKGROUND OF THE INVENTION

1. Field of the Invention

A composition and method for prevention and/or treatment of a respiratory infection in a subject before or after the onset of the disease.

2. Background

As civilization has progressed there has been a tendency to stay longer in closed and confined spaces due to work requirements or for physical comfort. Poor air quality is one of the major factors that produce respiratory infections in humans. The presence of microorganisms, toxins, and allergens is mainly due to poor ventilation, excess moisture, and improper cleaning and disinfection. The microorganisms responsible for respiratory problems include bacteria, fungi, and viruses present in these confined places. One such respiratory problem is sinusitis.

Sinusitis is caused by bacteria (e.g., streptococci, staphylococci, pneumococci, Haemophilus influenza), viruses (e.g., rhinovirus, influenza virus, parainfluenza virus), and/or fungi (e.g., Aspergillus, Dematiaceae, Mucoraceae, Penicillium sp.). The incidence of sinusitis (or inflammation of the sinuses) appears to be increasing. According to a survey of consumers and primary care physicians, 42 percent of people surveyed reported having at least one sinus infection in the last 12 months, compared to 33 percent the previous year. Apr. 2, 2002 issue of Sinus News, http://www.sinusnews.com/Articles2/Allergies-Colds-Sinusitis.html. Health care experts estimate that 37 million Americans are affected by sinusitis every year. Health care workers report 33 million cases of chronic sinusitis to the U.S. Centers for Disease Control and Prevention annually. Americans spend millions of dollars each year for medications that promise relief from their sinus symptoms.

Bacteria

Bacteria are the most common infectious agents in sinusitis. Most healthy people harbor bacteria, such as Streptococcus pneumoniae and Haemophilus influenzae, in their upper respiratory tracts with no problems until the body's defenses are weakened or drainage from the sinuses is blocked by a cold or other viral infection. Thus, bacteria that may have been living harmlessly can cause an acute sinus infection. Acute sinusitis in certain circumstances may progress into chronic sinusitis.

The bacteria most commonly implicated in sinusitis are the following:

1) Streptococcus pneumoniae (also called pneumococcal pneumonia or pneumococci). This bacterium is found in between 20% and 43% of adults with sinusitis.

2) H. influenzae (a common bacteria associated with many upper respiratory infections). This bacterium colonizes nearly half of all children by two years old. Studies have reported the presence of this bacteria in 22% to 35% of adult sinusitis patients.

3) Moraxella catarrhalis Over three-quarters of all children harbor this bacterium.

Less common bacterial culprits include other streptococcal strains (8% of adult cases) and Staphylococcus aureus (6% of adult cases).

The bacteria Streptococcus pneumoniae is said to be the leading cause of sinusitis, pneumonia, and ear infections in children. There is often pre-existing nasal colonization by pathogenic bacteria, especially penicillin-resistant pneumococci and nontypeable H. influenzae. The presence of these bacteria within the nasal cavity is common in children, especially those exposed to day care centers. Patients with particularly high fever or severe symptoms may have a superimposed acute bacterial infection. In patients with acute sinusitis, about 75% of maxillary sinus aspirates contain bacteria, usually S. pneumoniae, nontypeable H. influenzae, or Moraxella catarrhalis. In severe cases, Group A Streptococcus or Staphylococcus aureus may also be present. Similar bacteria are found in patients with subacute sinusitis. These organisms are also common in those with chronic sinusitis, although S. aureus, coagulase-negative staphylococci, alpha-hemolytic streptococci, and enteric bacilli are more common in this condition. Patients with chronic sinusitis usually have several species of anaerobes and one or more aerobic pathogens.

Mycoplasmas

Mycoplasmas are deemed bacteria but have a variety of differences relative to bacteria. One mycoplasma responsible for respiratory problems is Mycoplasma pneumoniae. These mycoplasmas are often found extracellular on mucosal surfaces.

Mycoplasma pneumoniae is a member of the class Mollicutes. Mycoplasmas are characterized by their unusually small genome (˜800 Kb) as well as their complete lack of a bacterial cell wall. Since mycoplasmas have both DNA and RNA present, they are deemed bacteria. Mycoplasmas are the smallest self-replicating organisms. Mycoplasma infections tend to be more chronic and indistinguishable on the basis of clinical symptoms alone. Thus, in many clinical settings, laboratory diagnosis is important for management.

Mycoplasma pneumoniae is a frequent cause of upper and lower respiratory tract infections. M. pneumoniae was first linked to respiratory infections in 1898 when Roux and Nocard isolated the organisms from bovine pleuropneumonia specimens. M. pneumoniae is currently thought to be responsible for both tracheobronchitis and primary a typical pneumonia. Overall, M. pneumoniae accounts for approximately 15-20% of all cases of pneumonia with higher rates reported among school children.

A combination of unique characteristics of mycoplasmas (lack of a cell wall, utilization of sterol in its membrane, and protein network which resembles an ancestral cytoskeleton) creates a different scenario for treatment of a mycoplasmal infection than other bacteria. The lack of a cell wall prevents the utilization of a B-lactam antibiotic, such as penicillin and cycloserine, because these antibiotics act specifically to disrupt the cell wall. The use of cholesterol in M. pneumoniae, however, allows for a different avenue for antibiotic therapies usually ineffective on bacteria. Though polyene antibiotics can be used against the cholesterols found in the membrane of mycoplasma, they can also act against the plasma membrane of the host cells.

Recent research has found a receptor on the surface of M. pneumoniae thought to be integral in the attachment to the host cell surface. This receptor can attach to a number of different cell types such as respiratory tract epithelia and red blood cells.

Viruses

The typical process leading to acute bacterial sinusitis actually starts with a flu or cold virus. Viruses themselves only rarely directly cause sinusitis. Instead, they produce inflammation and congestion in the nasal passages, called rhinitis, that leads to obstruction in the sinuses. This creates a hospitable environment for bacterial growth, which is the direct cause of sinus infection. In fact, rhinitis is the precursor to sinusitis in so many cases that expert groups now refer to sinusitis as rhinosinusitis. Viruses are directly implicated in only about 10% of sinusitis cases. Sinusitis occurring during the first week of upper respiratory infection is usually viral in origin. This self-limited condition is referred to as acute viral rhinosinusitis. Rhinovirus is a frequent cause of acute viral rhinitis.

Fungi

Sometimes, fungal infections can cause acute sinusitis. Although fungi are abundant in the environment, they usually are harmless to healthy people, indicating that the human body has a natural resistance to them. Fungi are uncommon causes of sinusitis, but the incidence of these infections is increasing. Fungal infections are suspected in people with sinusitis who also have diabetes, leukemia, AIDS, or other conditions that impair the immune system. Fungal infections can also occur in patients with healthy immune systems, but they are far less common than in impaired immune systems. Some people with fungal sinusitis have an allergic-type reaction to the fungi. Fungi involved in sinusitis are the following:

The fungus Aspergillus is the most common cause of all forms of fungal sinusitis.

Others include Curvularia, Bipolaris, Exserohilum, and Mucormycosis.

There have been a few reports of fungal sinusitis caused by Metarrhizium anisopliae, which is used in biological insect control.

There are four categories of fungal infections affecting the paranasal sinuses. These categories of fungal sinusitis are:

Acute or fulminant invasive fungal sinusitis. This infection is most likely to affect people with diabetes and compromised immune systems.

Chronic or indolent invasive fungal sinusitis. This form is generally found outside the U.S., most commonly in the Sudan and northern India.

Fungus ball (mycetoma). This fungal sinusitis is noninvasive and occurs usually in one sinus, most often the maxillary sinus.

Allergic fungal sinusitis. This form typically occurs because of an allergy to the fungus Aspergillus (rather than being caused by the fungus itself). In such cases, a peanut butter-like fungal growth occurs in the sinus cavities that may cause nasal passage obstruction and the erosion of the bones.

The offending fungi generally originate from the classes Zygomycetes (Mucor spp.) and Ascomycetes (Aspergillus spp.). Chronic sinusitis can develop into granulomatous chronic infection that may extend beyond the sinus walls. Allergic fungal sinusitis colonizes the sinuses of an atopic immunocompetent patient and acts as an allergen, eliciting an immune response. Fungal induced sinusitis most often is seen in immunosuppressed individuals such as those with AIDS, leukemia, lymphoma, or multiple myeloma, or in people with poor diabetes control. The Mayo Clinic Proceedings shows a report where 96% out of 210 patients with sinusitis had fungi. Fungal sinusitis is known as eosinophilic fungal rhinosinusitis (EFRS) or eosinophilic mucinous rhinosinusitis (EMRS).

Fungal infections can be very serious, and both chronic and acute fungal sinusitis require immediate treatment. Fungal ball is not invasive and is nearly always treatable. In some individuals, exposure to these fungi also can lead to asthma or to a lung disease resembling severe inflammatory asthma called allergic bronchopulmonary aspergillosis. Corticosteroid drugs are usually effective in treating this reaction. Immunotherapy is not helpful.

Treatment

Treatment and/or prevention of these infections have been in a number of ways. In addition to non-pharmaceutical treatments, such as hydration, nasal saline lavage, or surgery for abnormalities in the nasal cavity or sinuses, pharmaceutical treatments can be used.

Non-pharmaceutical methods of prevention include good hygiene (e.g., hand washing), healthy diet, and low stress.

Pharmaceutical treatments can include oral or topical decongestants, antipyretics, and analgesic medication. Zinc preparations using lozenges or nasal gels are now available as cold treatments. Vitamin C is often used for prevention or treatment. The herbal remedy echinacea is now commonly taken to prevent onset and ease symptoms of cold or flu.

Decongestants may be used for short-term treatment. They thicken secretions in the nasal passages, however, and may reduce the ability to clear out bacteria.

Nasal-delivery decongestants are applied directly into the nasal passages with a spray, gel, drops, or vapors. Nasal forms work faster than oral decongestants and have fewer side effects. They often require frequent administration, although long-acting forms are now available. The major hazard with nasal-delivery decongestants, particularly long-acting forms is a cycle of dependency and rebound effects.

Oral decongestants also come in many brands, which mainly differ in their ingredients. Certain adverse effects are more apt to occur in oral than nasal decongestants, and include the following: 1) agitation and nervousness, 2) drowsiness (particularly with oral decongestants and in combination with alcohol), 3) changes in heart rate and blood pressure, and 4) the need to avoid combinations of oral decongestants with alcohol or certain drugs, including monoamine oxidase inhibitors (MAOI) and sedatives.

Expectorants, which are drugs that cause mucus to be coughed up from the lungs and may help promote draining and reduce tissue swelling, are sometimes recommended for treatment of sinusitis. Expectorants generally contain ingredients that thin mucus secretions called mucolytics. Expectorants may cause drowsiness or nausea.

Many people take antipyretic and analgesic medications to reduce mild pain and fever related to respiratory infections. Adults most often choose aspirin, ibuprofen, or acetaminophen. It should be noted that some studies are suggesting that these anti-fever agents may actually reduce the body's immune response against cold and flu viruses and prolong symptoms.

In addition to decongestants, pain relievers, and expectorants, other remedies are available for people who suffer from nonbacterial sinusitis.

Antihistamines are the primary therapy for seasonal allergies, such as hay fever. They may also relieve congested sinuses that are not infected. People with bacterial infections in the nasal or sinus passages should not use antihistamines. These agents can thicken mucus secretions and may actually worsen bacterial infections.

Corticosteroid nasal sprays are also sometimes prescribed or recommended for patients with asthma or hay fever. They also can help reduce inflammation in the sinuses and relieve allergies but, like antihistamines, they are not effective in treating and may even worsen existing bacterial infection.

If decongestants or home remedies fail to improve sinusitis or if clear signs of infection or other complications are present, antibiotics are often prescribed. They are very effective in relieving symptoms and eliminating bacteria. Even after antibiotic treatments, between 10% and 25% of patients still complain of symptoms. In some cases, a stronger antibiotic may be needed. Most standard oral antibiotics require a seven to 10-day course with a tablet taken three or four times a day. Many people fail to complete such regimens. Patient non-compliance is very high with antibiotics. Failure to complete dosing may increase the risk for re-infection and also for development of antibiotic-resistant bacteria. Newer antibiotics are now available that can be taken once a day or for fewer days, although they tend to be expensive and may not be covered by some insurers.

The following are classes of antibiotics used for acute sinusitis under certain circumstances:

1) Beta-Lactams—The beta-lactam antibiotics share common chemical features and include penicillins and cephalosporins. Their primary action is to interfere with bacterial cell walls.

2) Penicillins—The most widely prescribed antibiotic for acute sinusitis has been amoxicillin (Amoxil®, Polymox®, Trimox®, Wymox®, or any generic formulation). This form of penicillin is both inexpensive and at one time was highly effective against the S. pneumoniae bacteria. Unfortunately, bacterial resistance to amoxicillin has. increased significantly, both among S. pneumoniae and H. influenzae. Amoxicillin-clavulanate (Augmentin®) is known as an augmented penicillin, which works against a wide spectrum of bacteria. Ampicillin, also a form of penicillin, is an equally inexpensive alternative to amoxicillin but requires more doses and has more severe gastrointestinal side effects than amoxicillin.

3) Cephalosporins—These agents have also become effective against S. pneumoniae. They are often classed in the following:

a) First generation include cephalexin (Keflex®), cefadroxil (Duricef®, Ultracef™), and cefaclor (Ceclor®). These are not used for upper respiratory infections, although cefaclor may have some effectiveness against effective against H. influenzae.

b) Second and third generation include cefuroxime (Ceftin®), cefpodoxime (Vantin®), loracarbef (Lorabid®), cefditoren (Spectracef®), cefixime (Suprax®), and ceftibuten (Cedex). These are effective against a wide spectrum of bacteria. Among the cephalosporins, cefpodoxime, and cefuroxime have the best record to date for coverage against bacteria that infect the upper respiratory tract. They are not effective, however, against S. pneumoniae bacteria that have developed resistance to penicillin.

4) Fluoroquinolones—Fluoroquinolones (also simply called quinolones) interfere with the bacteria's genetic material so they cannot reproduce. They include ciprofloxacin (Cipro®), levofloxacin (Levaquin®), sparfloxacin (Zagam®), gemifloxacin (Factive®), gatifloxacin (Tequin®), moxifloxacin (Avelox®), and ofloxacin (Floxin®). The newer fluoroquinolones, particularly levofloxacin, gatifloxacin, moxifloxacin, and sparfloxacin are currently the most effective agents against the common bacteria that cause sinusitis. Some of the newer fluoroquinolones also only need to be taken once a day, which makes patient compliance easier.

5) Macrolides and Azalides—Macrolides and azalides are antibiotics that also affect the genetics of bacteria. They include erythromycin, azithromycin (Zithromax®), clarithromycin (Biaxin®), and roxithromycin (Rulid®). These antibiotics are effective against S. pneumoniae and M catarrhalis, but there is increasing bacterial resistance to these agents. Except for erythromycin they are effective against H. influenzae. Clarithromycin has anti-inflammatory actions and might be especially useful for certain patients with chronic sinusitis. A new once-a-day formulation (Biaxin® XL) is now available.

6) Lincosamide—Lincosamides prevent bacteria from reproducing. The most common lincosamide is clindamycin (Cleocin®). This antibiotic is useful against many S. pneumoniae bacteria but not against H. influenzae.

7) Tetracyclines—Tetracyclines inhibit bacterial growth. They include doxycycline, tetracycline, and minocyclin. They can be effective against S. pneumoniae and M. catarrhalis, but bacteria that are resistant to penicillin are also often resistant to doxycycline. Tetracyclines have unique side effects among antibiotics, including skin reactions to sunlight, possible burning in the throat, and tooth discoloration.

8) Trimethoprim-Sulfamethoxazole—Physicians commonly prescribe trimethoprim-sulfamethoxazole (Bactrim, Cotrim, Septra®) for sinusitis. It is less expensive than amoxicillin and particularly useful for adults with mild sinusitis who are allergic to penicillin. It is no longer effective, however against certain streptococcal strains. It should not be used in patients whose infections occurred after dental work or in patients allergic to sulfa drugs. Allergic reactions can be very serious.

Most antibiotics have the following side effects (although specific antibiotics may have other side effects or fewer of the standard ones).

1) The most common side effect for nearly all antibiotics is gastrointestinal distress.

2) Antibiotics double the risk for vaginal infections in women.

3) Allergic reactions can also occur with all antibiotics but are most common with medications derived from penicillin or sulfa. These reactions can range from mild skin rashes to rare but severe, even life-threatening anaphylactic shock.

4) Certain drugs, including some over-the-counter medications, interact with antibiotics.

Of great concern is the emergence of common bacteria strains that are now resistant to many standard antibiotics. Among the bacteria are those that cause serious respiratory infections, including pneumonia. Although new powerful antibiotics continue to designed, they are expensive and are also prone to resistance eventually.

Acute sinusitis is often treated with decongestants, antibiotics, and pain relievers. Rhinovirus is a frequent cause of acute viral rhinitis.

Acute bacterial sinusitis may also occur. Appropriate antimicrobial treatment and close follow up care are critically important. If criteria suggesting bactermeia or intracraneal infection are not present, oral antimicrobials are useful for acute sinusitis. Treatment choices are directed toward S. pneumonia, H. influenzae, and Moraxella.

Chronic sinusitis is more difficult. Doctors often find it difficult to treat chronic sinusitis successfully, realizing that symptoms persist even after taking antibiotics for a long period. Doctors commonly prescribe steroid nasal sprays to reduce inflammation in chronic sinusitis. A doctor may prescribe oral steroids, such as prednisone, in severe chronic sinusitis. When medical treatment fails, surgery may be the only alternative for treating chronic sinusitis.

Sinusitis caused by severe fungal infections is a medical emergency. Treatment is aggressive surgery and high-dose antifungal chemotherapy with a drug such as amphotericin B. The use of oxygen administered at high pressure (hyperbaric oxygen) is showing promise as additional therapy for potentially deadly fungal infections.

Vaccines are also being used for respiratory illness. Vaccines against influenza currently employ inactivated viruses to produce an immune response that will then attack the active virus. A live but weakened intranasal vaccine (FluMist®) should be available soon. The vaccine boosts the specific immune factors in the mucous membranes of the nose that fight off the actual viral infections. It is employed using a nasal spray and in one study provided protection against the flu in up to 93% of children. At this time, vaccines must be redesigned each year to match the current strain. This is because both influenza A and B viral strains undergo changes over time (known as antigenic drift or shift), so a vaccine that works one year may not work the next. Influenza A is a particular problem because it can infect other species, such as pigs or chickens, and undergo major genetic reassortments. Influenza B viruses tend to be more stable than influenza A viruses, but they too vary. The vaccines may be slightly less effective in the elderly, the very young, and patients with certain chronic diseases than in healthy young adults. The vaccinations protect against influenza in between 70% and 100% of healthy adults when the virus and the vaccine are well matched. In the absence of a match and among the elderly and children, they are protective in 30% to 60% of people. Possible negative responses include the following:

Newer vaccines contain very little egg protein, but an allergic reaction still may occur in people with strong allergies to eggs.

Almost a third of people who receive the influenza vaccine develop redness or soreness at the injection site for one or two days afterward.

Other side effects include mild fatigue and muscle aches and pains; they tend to occur between six and 12 hours after the vaccination and last up to two days. It should be noted that these symptoms are not influenza itself but an immune response to the virus proteins in the vaccine.

Some studies have reported more severe asthma symptoms in children with the lung condition. More research is needed to confirm or refute these results.

A nasal spray, tremacamra, is under investigation for treating colds. It contains a genetically engineered compound that resembles a natural molecule called ICAM-1, which is located in human cells and attaches to rhinoviruses that are present in the nasal passages. The similar tremacamra tricks the virus into attaching to it rather than to the ICAM-1 receptor, thereby preventing the virus from affecting human cells. Studies suggest that it reduces the severity of a cold, although its effect on duration is not clear.

Several other drugs are being studied for prevention and treatment of colds. One, pleconaril, inhibits viral attachment and is also showing promise.

These previous compositions and methods have drawbacks which are discussed above. These include for example, resistance to antibiotics, allergic reactions, and various side effects.

Terpenes are widespread in nature, mainly in plants as constituents of essential oils. Their building block is the hydrocarbon isoprene (C₅H₈)ₙ. Terpenes have been found to be effective and nontoxic dietary anti-tumor agents which act through a variety of mechanisms of action (Crowell, P. L. and M. N. Gould, 1994. Chemoprevention and therapy of cancer by d-limonene. Crit. Rev. Oncog. 5(1): 1-22; Crowell, P. L., S. Ayoubi and Y. D. Burke, 1996. Antitumorigenic effects of limonene and perillyl alcohol against pancreatic and breast cancer. Adv. Exp. Med. Biol. 401: 131-136). Terpenes, i.e., geraniol, tocotrienol, perillyl alcohol, b-ionone, and d-limonene, suppress hepatic HMG-COA reductase activity, a rate limiting step in cholesterol synthesis, and modestly lower cholesterol levels in animals (Elson, C. E. and S. G. Yu, 1994. The chemoprevention of cancer by mevalonate-derived constituents of fruits and vegetables. J. Nutr. 124: 607-614). D-limonene and geraniol reduced mammary tumors (Elegbede, J. A., C. E. Elson, A. Qureshi, M. A. Tanner and M. N. Gould, 1984. Inhibition of DMBA-induced mammary cancer by monoterpene d-limonene. Carcinogenesis 5(5): 661-664; Elegbede, J. A., C. E. Elson, A. Qureshi, M. A. Tanner and M. N. Gould, 1986. Regression of rat primary mammary tumors following dietary d-limonene. J. Natl. Cancer Inst. 76(2): 323-325; Karlson, J., A. K. Borg, R. Unelius, M. C. Shoshan, N. Wilking, U. Ringborg and S. Linder, 1996. Inhibition of tumor cell growth by monoterpenes in vitro: evidence of a Ras-independent mechanism of action. Anticancer Drugs 7(4): 422-429) and suppressed the growth of transplanted tumors (Yu, S. G., P. J. Anderson and C. E. Elson, 1995. The efficacy of B-ionone in the chemoprevention of rat mammary carcinogenesis. J. Agri. Food Chem. 43: 2144-2147).

Terpenes have also been found to inhibit the in vitro growth of bacteria and fungi (Chaumont J. P. and D. Leger, 1992. Campaign against allergic moulds in dwellings. Inhibitor properties of essential oil geranium “Bourbon”, citronellol, geraniol and citral. Ann Pharm Fr 50(3): 156-166; Moleyar, V. and P. Narasimham, 1992. Antibacterial activity of essential oil components. Int. J. Food Microbiol. 16(4): 337-342; and Pattnaik, S., V. R. Subramanyan, M. Bapaji and C. R. Kole, 1997. Antibacterial and antifungal activity of aromatic constituents of essential oils. Microbios. 89(358): 39-46) and some internal and external parasites (Hooser, S. B., V. R. Beasly and J. J. Everitt, 1986. Effects of an insecticidal dip containing d-limonene in the cat. J. Am. Vet. Med. Assoc. 189(8): 905-908). Geraniol was found to inhibit growth of Candida albicans and Saccharomyces cerevisiae strains by enhancing the rate of potassium leakage and disrupting membrane fluidity (Bard, M., M. R. Albert, N. Gupta, C. J. Guuynn and W. Stillwell, 1988. Geraniol interferes with membrane functions in strains of Candida and Saccharomyces. Lipids 23(6): 534-538). B-ionone has antifungal activity which was determined by inhibition of spore germination, and growth inhibition in agar (Mikhlin, E. D., V. P. Radina, A. A. Dmitrossky, L. P. Blinkova and L. G. Button, 1983. Antifungal and antimicrobial activity of some derivatives of beta-ionone and vitamin A. Prikl. Biokhim. Mikrobiol. 19: 795-803; Salt, S. D., S. Tuzun and J. Kuc, 1986. Effects of B-ionone and abscisic acid on the growth of tobacco and resistance to blue mold. Mimicry the effects of stem infection by Peronospora tabacina. Adam. Physiol. Molec. Plant Path. 28: 287-297). Teprenone (geranylgeranylacetone) has an antibacterial effect on H. pylori (Ishii, E., 1993. Antibacterial activity of teprenone, a non water-soluble antiulcer agent, against Helicobacter pylori. Int. J. Med. Microbiol. Virol. Parasitol. Infect. Dis. 280(1-2): 239-243). Rosanol, a commercial product with 1% rose oil, has been shown to inhibit the growth of several bacteria (Pseudomonas, Staphylococus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim and D. H. Kim, 1998. Anti-Helicobacter pylori activity of herbal medicines. Biol. Pharm. Bull. 21(9) 990-992). Extracts from cashew apple, anacardic acid, and (E)-2-hexenal have shown bactericidal effect against H. pylori.

Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall, and C. Wei, 1995. Antibacterial activity of some essential oil components against five foodborne pathogens. J. Agric. Food Chem. 43: 2839-2845). Diterpenes, i.e., trichorabdal A (from R. Trichocarpa), has shown a very strong antibacterial effect against H. pylori (Kadota, et al., 1997).

There may be different modes of action of terpenes against microorganisms; they could (1) interfere with the phospholipid bilayer of the cell membrane, (2) impair a variety of enzyme systems (HMG-reductase), and (3) destroy or inactivate genetic material.

For the above reasons, and others, the present invention provides additional methods for controlling respiratory infections that avoid the drawbacks of previous methods.

### SUMMARY OF THE INVENTION

In accordance with the purpose(s) of this invention, as embodied and broadly described herein, this invention relates to prevention and/or treatment of infections, especially respiratory infections.

The invention is related to the field of anti-infectives. The present invention provides compositions and methods for treating and/or preventing a respiratory infection that avoid some drawbacks found in previous methods.

The present invention provides a composition for treating and/or preventing an infection, especially a respiratory infection, in a subject comprising an effective amount of at least one effective terpene. The composition can be a solution, especially a true solution. The composition can further comprise a carrier, e.g., water. The composition can further comprise a surfactant and water.

The composition may be a solution of terpene and water.

The composition of invention can comprise a mixture of different terpenes or a terpene-liposome (or other vehicle) combination.

The terpene of the composition can comprise, for example, citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof.

The composition is effective against various infective agents including bacteria, viruses, mycoplasmas, and/or fungi.

A composition for treating and/or preventing a respiratory infection in a subject comprising a true solution comprising an effective amount of at least one effective terpene and water is also disclosed.

Further shown is a pharmaceutical composition for treating and/or preventing a respiratory infection comprising an effective amount of an effective terpene and a pharmaceutically acceptable carrier.

A method for preventing and/or treating a respiratory infection comprising administering a composition comprising an effective amount of an effective terpene to a subject is also disclosed. The administration of the method can be by inhalation of the composition, for example, by the inhalation of an aerosol solution containing a single bioactive terpene, a bioactive terpene mixture, or a liposome-terpene(s) composition with or without a surfactant.

The methods are practiced using the compositions of the present invention.

The composition can be made by mixing an effective amount of an effective terpene and water. The mixing can be done at a solution-forming shear until formation of a true solution of the terpene and water, the solution-forming shear may be by high shear or high pressure blending or agitation.

The invention includes a method for making a terpene-containing composition effective for preventing and/or treating infections comprising mixing a composition comprising a terpene and water at a solution-forming shear until a true solution of the terpene is formed.

The invention is further a method for making a terpene-containing composition effective for preventing and/or treating infections comprising adding terpene to water, and mixing the terpene and water under solution-forming shear conditions until a true solution of terpene and water forms.

A method of prevention and/or treatment of a respiratory infection comprising inhalation by a subject of an aerosol solution comprising a single effective terpene, an effective terpene mixture, or a liposome-terpene(s) composition.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE PREFERRED EMBODIMENTS

Before the present compounds, compositions, articles, devices, and/or methods are disclosed and described, it is to be understood that this invention is not limited to specific synthetic methods. It is also to be understood that the terminology used herein is for the purpose of describing particular aspects only and is not intended to be limiting.

In this specification and in the claims which follow, reference will be made to a number of terms which shall be defined to have the following meanings:

Definitions

It must be noted that, as used in the specification and the appended claims, the singular forms “a,” “an,” and “the” include plural referents unless the context clearly dictates otherwise. Thus, for example, reference to “an aerosol” includes mixtures of aerosols, reference to “a terpene” includes mixtures of two or more such terpenes, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by volume, of a particular element or component in a composition or article, denotes the volume relationship between the element or component and any other elements or components in the composition or article for which a part by volume is expressed. Thus, in a composition containing 2 parts by volume of component X and 5 parts by volume component Y, X and Y are present at a volume ratio of 2:5, and are present in such ratio regardless of whether additional components are contained in the composition.

A volume percent of a component, unless specifically stated to the contrary, is based on the total volume of the formulation or composition in which the component is included.

“Optional” or “optionally” means that the subsequently described event or circumstance may or may not occur, and that the description includes instances where said event or circumstance occurs and instances where it does not. For example, the phrase “optionally surfactant” means that the surfactant may or may not be added and that the description includes both with a surfactant and without a surfactant where there is a choice.

By the term “effective amount” of a compound or property as provided herein is meant such amount as is capable of performing the function of the compound or property for which an effective amount is expressed, such as a non-toxic but sufficient amount of the compound to provide the desired function, i.e., anti-infective. As will be pointed out below, the exact amount required will vary from subject to subject, depending on the subject, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

By the term “effective terpene” is meant a terpene which is effective against the particular infective agent of interest.

By “pharmaceutically acceptable” is meant a material that is not biologically or otherwise undesirable, i.e., the material may be administered to an individual subject along with the selected composition without causing any undesirable biological effects or interacting in a deleterious manner with any of the other components of the composition in which it is contained.

As used throughout, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.) and birds. In one aspect, the subject is a mammal, such as a primate or a human.

By the term “true solution” is meant a solution (essentially homogeneous mixture of a solute and a solvent) in contrast to an emulsion or suspension. A visual test for determination of a true solution is a clear resulting liquid. If the mixture remains cloudy, or otherwise not clear, it is assumed that the mixture formed is not a true solution but instead a mixture such as an emulsion or suspension.

Poor air quality is one of the major factors that produce respiratory infections in humans. The presence of microorganisms, toxins, and allergens is mainly due to poor ventilation, excess moisture, and improper cleaning and disinfection. The present invention has the capacity of reducing the incidences of and treating respiratory infections. The composition comprises terpenes, which can be naturally-occurring chemicals that are found in plants, which are generally recognized as safe (GRAS) by the FDA. An aspect of this invention is that due to the mechanism of action, such as basic interference with cholesterol, terpenes do not generate microbial resistance. There are antimicrobial products containing terpenes, basically in the form of essential oils, but we have found that not all components of the essential oils are biocides.

Another aspect of the present invention is that by varying the concentration of terpenes different specificity and biocidal effect can be achieved and that by combining two or more terpenes in the same solution a synergistic effect can be obtained. A further aspect of this invention is that the terpenes and surfactant used are generally recognized as safe (GRAS) by the FDA. An additional aspect of this invention is that we can tailor the formulation and obtain biocidal effect over a single type microorganism or change the formulation and eliminate all types of microorganisms.

Applying one of the formulations of the present invention in spray form into the nasal cavities reduces the amount of microorganism responsible of infections like Aspergillius and Stachybotrys (fungi). These microorganisms are responsible for the majority of respiratory infections present in immuno-deficient patients and children. Several formulations can be obtained by utilizing biocidal terpenes without departing from the principle of the present inventions.

Formulations can vary not only in the concentration of terpenes but also in the type of surfactant used, if any. This invention can be readily be mixed with other types of nasal delivery medications. Another advantage of the present invention is that the terpenes present in the formulation can reach all areas of the respiratory system including the lungs.

We have found that higher concentrations of certain terpenes can be irritating to the nasal passages, and that by reducing or eliminating these terpenes in the formulation we still have the benefit of the other terpenes. The terpenes that have been tested to date in the present invention include citral, carvone, eugenol, and b-ionone. All of them have biocidal properties; and other biocidal terpenes can be utilized without departing from the scope of the present invention.

We have observed that the terpenes used in this invention can be targeted to different microorganisms. We have been able to prove the effectiveness of the present invention against microorganisms that are of importance for humans and animals. Also, the effective terpene dose varies depending on the organism we are interested in eliminating.

This invention can be modified in several ways by adding or deleting from the formulation the type of terpene and surfactant.

The present invention includes methods of making the compositions and methods of using the compositions.

Composition(s)

The compositions of the present invention comprise isoprenoids. More specifically, the compositions of the present invention comprise terpenoids. Even more specifically, the compositions of the present invention comprise terpenes. Terpenes are widespread in nature, mainly in plants as constituents of essential oils. Terpenes are unsaturated aliphatic cyclic hydrocarbons. Their building block is the hydrocarbon isoprene (C₅H₈)ₙ. A terpene is any of various unsaturated hydrocarbons, such as C₁₀H₁₆, found in essential oils, oleoresins, and balsams of plants, such as conifers. Some terpenes are alcohols (e.g., menthol from peppermint oil), aldehydes (e.g., citronellal), or ketones.

Terpenes have been found to be effective and nontoxic dietary antitumor agents, which act through a variety of mechanisms of action. Crowell, P. L. and M. N. Gould, 1994. Chemoprevention and Therapy of Cancer by D-limonene, Crit. Rev. Oncog. 5(1): 1-22; Crowell, P. L., S. Ayoubi and Y. D. Burke, 1996, Antitumorigenic Effects of Limonene and Perillyl Alcohol Against Pancreatic and Breast Cancer, Adv. Exp. Med. Biol. 401: 131-136. Terpenes, i.e., geraniol, tocotrienol, perillyl alcohol, b-ionone and d-limonene, suppress hepatic HMG-COA reductase activity, a rate limiting step in cholesterol synthesis, and modestly lower cholesterol levels in animals. Elson C. E. and S. G. Yu, 1994, The Chemoprevention of Cancer by Mevalonate-Derived Constituents of Fruits and Vegetables, J. Nutr. 124: 607-614. D-limonene and geraniol reduced mammary tumors (Elgebede, J. A., C. E. Elson, A. Qureshi, M. A. Tanner and M. N. Gould, 1984, Inhibition of DMBA-Induced Mammary Cancer by Monoterpene D-limonene, Carcinogensis 5(5): 661-664; Elgebede, J. A., C. E. Elson, A. Qureshi, M. A. Tanner and M. N. Gould, 1986, Regression of Rat Primary Mammary Tumors Following Dietary D-limonene, J. Nat'l Cancer Institute 76(2): 323-325; Karlson, J., A. K. Borg, R. Unelius, M. C. Shoshan, N. Wilking, U. Ringborg and S. Linder, 1996, Inhibition of Tumor Cell Growth By Monoterpenes In Vitro: Evidence of a Ras-Independent Mechanism of Action, Anticancer Drugs 7(4): 422-429) and suppressed the growth of transplanted tumors (Yu, S. G., P. J. Anderson and C. E. Elson, 1995, The Efficacy of B-ionone in the Chemoprevention of Rat Mammary Carcinogensis, J. Angri. Food Chem. 43: 2144-2147).

Terpenes have also been found to inhibit the in vitro growth of bacteria and fungi (Chaumont J. P. and D. Leger, 1992, Campaign Against Allergic Moulds in Dwellings, Inhibitor Properties of Essential Oil Geranium “Bourbon,” Citronellol, Geraniol and Citral, Ann. Pharm. Fr 50(3): 156-166), and some internal and external parasites (Hooser, S. B., V. R. Beasly and J. J. Everitt, 1986, Effects of an Insecticidal Dip Containing D-limonene in the Cat, J. Am. Vet. Med. Assoc. 189(8): 905-908). Geraniol was found to inhibit growth of Candida albicans and Saccharomyces cerevisiae strains by enhancing the rate of potassium leakage and disrupting membrane fluidity (Bard, M., M. R. Albert, N. Gupta, C. J. Guuynn and W. Stillwell, 1988, Geraniol Interferes with Membrane Functions in Strains of Candida and Saccharomyces, Lipids 23(6): 534-538). B-ionone has antifungal activity which was determined by inhibition of spore germination and growth inhibition in agar (Mikhlin E. D., V. P. Radina, A. A. Dmitrossky, L. P. Blinkova, and L. G. Button, 1983, Antifungal and Antimicrobial Activity of Some Derivatives of Beta-Ionone and Vitamin A, Prikl Biokhim Mikrobiol, 19: 795-803; Salt, S. D., S. Tuzun and J. Kuc, 1986, Effects of B-ionone and Abscisic Acid on the Growth of Tobacco and Resistance to Blue Mold, Mimicry the Effects of Stem Infection by Peronospora Tabacina, Adam Physiol. Molec. Plant Path 28:287-297). Teprenone (geranylgeranylacetone) has an antibacterial effect on H. pylori (Ishii, E., 1993, Antibacterial Activity of Terprenone, a Non Water-Soluble Antiulcer Agent, Against Helicobacter Pylori, Int. J. Med. Microbiol. Virol. Parasitol. Infect. Dis. 280(1-2): 239-243). Solutions of 11 different terpenes were effective in inhibiting the growth of pathogenic bacteria in in vitro tests; levels ranging between 100 ppm and 1000 ppm were effective. The terpenes were diluted in water with 1% polysorbate 20 (Kim, J., M. Marshall and C. Wei, 1995, Antibacterial Activity of Some Essential Oil Components Against Five Foodborne Pathogens, J. Agric. Food Chem. 43: 2839-2845). Diterpenes, i.e., trichorabdal A (from R. Trichocarpa), have shown a very strong antibacterial effect against H. pylori (Kadota, S., P. Basnet, E. Ishii, T. Tamura and T. Namba, 1997, Antibacterial Activity of Trichorabdal A from Rabdosia Trichocarpa Against Helicobacter Pylori, Zentralbl. Bakteriol 287(1): 63-67).

Rosanol, a commercial product with 1% rose oil, has been shown to inhibit the growth of several bacteria (Pseudomona, Staphylococus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae, E. A., M. J. Han, N. J. Kim, and D. H. Kim, 1998, Anti-Helicobacter Pylori Activity of Herbal Medicines, Biol., Pharm. Bull. 21(9) 990-992). Extracts from cashew apple, anacardic acid, and (E)-2-hexenal, have shown bactericidal effect against H. pylori. There may be different modes of action of terpenes against microorganism; they could (1) interfere with the phospholipid bilayer of the cell membrane, (2) impair a variety of enzyme systems (HMG-reductase), and (3) destroy or inactivate genetic material.

It is believed that due to the modes of action of terpenes being so basic, e.g., blocking of cholesterol, that infective agents will not be able to build a resistance to terpenes.

Terpenes, which are Generally Recognized as Safe (GRAS) have been found to inhibit the growth of cancerous cells, decrease tumor size, decrease cholesterol levels, and have a biocidal effect on microorganisms in vitro. Owawunmi, G. O., 1989, Evaluation of the Antimicrobial Activity of Citral, Letters in Applied Microbiology 9(3): 105-108, showed that growth media with more than 0.01% citral reduced the concentration of E. coli, and at 0.08% there was a bactericidal effect. Barranx, A. M. Barsacq, G. Dufau, and J. P. Lauilhe, 1998, Disinfectant or Antiseptic Composition Comprising at Least One Terpene Alcohol and at Lease One Bactericidal Acidic Surfactant, and Use of Such a Mixture, U.S. Pat. No. 5,673,468, teach a terpene formulation, based on pine oil, used as a disinfectant or antiseptic cleaner. Koga, J. T. Yamauchi, M. Shimura, Y. Ogasawara, N. Ogasawara and J. Suzuki, 1998, Antifungal Terpene Compounds and Process for Producing the Same, U.S. Pat. No. 5,849,956, teach that a terpene found in rice has antifungal activity. Iyer, L. M., J. R. Scott, and D. F. Whitfield, 1999, Antimicrobial Compositions, U.S. Pat. No. 5,939,050, teach an oral hygiene antimicrobial product with a combination of 2 or 3 terpenes that showed a synergistic effect. Several U.S. patents (U.S. Pat. Nos. 5,547,677, 5,549,901, 5,618,840, 5,629,021, 5,662,957, 5,700,679, 5,730,989) teach that certain types of oil-in-water emulsions have antimicrobial, adjuvant, and delivery properties.

Terpenes are widespread in nature. Their building block is the hydrocarbon isoprene (C₅H₈)ₙ. Examples of terpenes include citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, and linalool.

An effective terpene of the composition can comprise, for example, citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, linalool, or mixtures thereof. More specifically, the terpene can comprise citral, carvone, eugenol, b-ionone, eucalyptus oil, or mixtures thereof.

The composition can comprise an effective amount of the terpene. By the term “effective amount” of a composition as provided herein is meant a nontoxic but sufficient amount of the composition to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The composition can comprise between about 100 ppm and about 2000 ppm of the terpene, specifically 100, 250, 500, or 1000 ppm.

A composition of the present invention comprises an effective amount of an effective terpene. An effective (i.e., anti-infective) amount of the effective terpene is the amount that produces a desired effect, i.e., prevention and/or treatment of an infection. This is the amount that will reach the necessary locations of the subject at a concentration which will kill the infective agent. Though less than a full kill may be effective, this will likely have little value to an end user since it is relatively easy to adjust the amount to achieve a full kill. If there were an instance where the amount for a full kill was very close to the toxic amount, an amount that achieves a stable population or stasis of the infective agent may be sufficient to prevent disease progression. An effective (i.e., anti-infective) terpene is one which produces the desired effect, i.e., prevention or treatment of a respiratory infection, against the particular infective agent(s) with the potential to infect or which have infected the subject(s).

The most effective terpenes can be the C₁₀H₁₆ terpenes. The more active terpenes for this invention can be the ones which contain oxygen. It is preferred for regulatory and safety reasons that at least food grade terpenes (as defined by the U.S. FDA) be used.

The composition can comprise a single terpene, more than one terpene, a liposome-terpene combination, or combinations thereof. Mixtures of terpenes can produce synergistic effects.

All classifications of natural or synthetic terpenes will work in this invention, e.g., monoterpenes, sesquiterpenes, diterpenes, triterpenes, and tetraterpenes. Examples of terpenes that can be used in the present invention are citral, pinene, nerol, b-ionone, geraniol, carvacrol, eugenol, carvone, terpeniol, anethole, camphor, menthol, limonene, nerolidol, farnesol, phytol, carotene (vitamin A₁), squalene, thymol, tocotrienol, perillyl alcohol, borneol, myrcene, simene, carene, terpenene, and linalool. The list of exempted terpenes found in EPA regulation 40 C.F.R. Part 152 is incorporated herein by reference in its entirety. The terpenes may also be known by their extract or essential oil names, such as lemongrass oil (contains citral).

Citral, for example citral 95, is an oxygenated C₁₀H₁₆ terpene, C₁₀H₁₆O CAS No. 5392-40-5 3,7-dimethyl-2,6-octadien-1-al.

Plant extracts or essential oils containing terpenes can be used in the compositions of this invention as well as the more purified terpenes.

Terpenes are readily commercially available or can be produced by various methods known in the art, such as solvent extraction or steam extraction/distillation. Natural or synthetic terpenes are expected to be effective in the invention. The method of acquiring the terpene is not critical to the operation of the invention.

The liposome-terpene(s) combination comprises encapsulation of the terpene, attachment of the terpene to a liposome, or is a mixture of liposome and terpene. Alternatively, vehicles other than liposomes may be used, such as microcapsules or microspheres. If the liposome or encapsulating vehicle serves as a time release device and may not be taken up by the cells of the subject, the size and structure of the vehicle can be determined by one of skill in the art based on the desired release amounts and timing. If the liposome or encapsulating vehicle serves as a vehicle in which to get the composition into the cells of the subject, the size and structure of the vehicle can be determined by one of skill in the art based on the sizes which the desired cells will engulf or otherwise bring the composition into the cell. The forms of the compositions that are not taken up by the cells can be used as extracelluar treatments, for example, on the mucosa.

It is known to one of skill in the art how to produce a liposome or other encapsulating vehicle. For example, an oil-in-oil-in water composition of liposome-terpene may be used.

The composition can further comprise additional ingredients. For example, water (or alternatively, any bio-compatible or pharmaceutically acceptable dilutant or carrier), a surfactant, preservative, or stabilizer.

The surfactant can be non-ionic, cationic, or anionic. Examples of surfactant include polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, Tween®, Span® 20, Span® 40, Span® 60, Span® 80, or mixtures thereof.

The composition can comprise 1 to 99% by volume terpenes and 0 to 99% by volume surfactant. More specifically the composition can comprise about 100 to about 2000 ppm terpenes and about 10% surfactant.

The concentration of terpene in the composition is an anti-infective amount. This amount can be from about an infective agent controlling level (e.g., about 100 ppm) to about a level with side effects or possibly even a level toxic to the subject's cells (e.g., about 2000 ppm generally caused irritation in humans, though the level may be cell or subject specific). This amount can vary depending on the terpene(s) used, the form of terpene (e.g., liposome-terpene), the infective agent targeted, and other parameters that would be apparent to one of skill in the art. One of skill in the art would readily be able to determine an anti-infective amount for a given application based on the general knowledge in the art and the procedures in the Examples given below.

Specific compositions can include e.g.,

bacteria and fungi—1000 ppm terpenes in standard 0.9% saline with 50% 1-carvone, 30% eugenol, 10% purified eucalyptus oil, and 10% Tween® 80;

for mold—1000 ppm terpenes in water 100% citral or 95% citral and 5% Tween(80; or

for mycoplasma—125 ppm or 250 ppm in PBS 95% b-ionone and 5% Tween® 80.

Concentrations of terpene of 80, 90, 100, 110, 125, 130, 140, 150, 160, 175, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1250, 1375, 1425, 1500, 1600, 1750, or 2000 ppm can be used as effective concentrations in the compositions and methods of the current invention.

Concentrations of any other ingredients or components can also be readily determined by one of skill in the art using methods known in the art and demonstrated below.

Terpenes have a relatively short life span of approximately 28 days once exposed to oxygen (e.g., air). Terpenes will decompose to CO₂ and water. This decomposition or break down of terpenes is an indication of the safety and environmental friendliness of the compositions and methods of the invention.

The LD₅₀ in rats of citral is approximately 5 g/kg. This also is an indication of the relative safety of these compounds.

A stable suspension of citral can be formed up to about 2500 ppm. Citral can be made into a solution at up to about 500 ppm.

Of the terpenes tested, citral has been found to form a solution at the highest concentration level. Citral will form a solution in water up to about 1000 ppm and will lyse human erythrocytes at approximately 1000 ppm.

At sufficiently high levels of terpene, a terpene acts as a solvent and will lyse cell walls. Example 10 shows the levels that will lyse red blood cells.

A composition comprising a terpene, water, and a surfactant forms a suspension of the terpene in the water. Some terpenes may need a surfactant to form a relatively homogeneous mixture with water.

A composition comprising a “true” solution of a terpene is desired in order to minimize additional components which may cause undesired effects. A method for making a true solution comprising a terpene is described below.

The composition(s) of the present invention are effective against most infective agents. Examples of infective agents include fungi, viruses, bacteria, and mycoplasmas.

The terpenes, surfactants, or other components of the invention may be readily purchased or synthesized using techniques generally known to synthetic chemists. Methods for making specific and exemplary compositions of the present invention are described in detail in the Examples below.

The compositions of the invention may be conveniently formulated into pharmaceutical compositions composed of one or more of the compositions in association with a pharmaceutically acceptable carrier. See, e.g., Remington's Pharmaceutical Sciences, latest edition, by E. W. Martin Mack Pub. Co., Easton, Pa., which discloses typical carriers and conventional methods of preparing pharmaceutical compositions that may be used in conjunction with the preparation of formulations of the present invention and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents, and the like, in addition to the composition of choice. Pharmaceutical compositions can also include one or more active ingredients such as antimicrobial agents, antiinflammatory agents, anesthetics, and the like.

The pharmaceutical compositions described herein can be administered to the subject in a number of ways depending on whether local or systemic treatment is desired, and on the area to be treated. Thus, for example, a pharmaceutical composition described herein can be administered as an aerosol to the surface of the nasal mucosa. Moreover, a pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal, and intratracheal routes.

Parenteral administration, if used, is generally characterized by injection. Injectables can be prepared in conventional forms, either as liquid solutions or suspensions, solid forms suitable for solution or suspension in liquid prior to injection, or as emulsions. The amount of composition administered will, of course, be dependent on the subject being treated, the subject's weight, the manner of administration and the judgment of the prescribing physician.

Depending on the intended mode of administration, the pharmaceutical compositions may be in the form of solid, semi-solid, or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of the selected composition in combination with a pharmaceutically acceptable carrier and, in addition, may include other medicinal agents, pharmaceutical agents, carriers, adjuvants, diluents, etc.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the-like. Liquid pharmaceutically administrable compositions can, for example, be prepared by dissolving, dispersing, etc., an effective terpene as described herein and optional pharmaceutical adjuvants in an excipient, such as, for example, water, saline aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents, and the like, for example, sodium acetate, sorbitan monolaurate, triethanolamine sodium acetate, triethanolamine oleate, etc. Actual methods of preparing such dosage forms are known, or will be apparent, to those skilled in this art; for example see Remington's Pharmaceutical Sciences, referenced above.

For oral administration, if used, fine powders or granules may contain diluting, dispersing, and/or surface active agents and may be presented in water or in a syrup, in capsules or sachets in the dry state, or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are generally preferred oral administration forms in the art, and these may be coated.

Parental administration, if used, is generally characterized by injection. Injectables can be prepared in conventional forms, either as liquid solutions or suspensions, solid forms suitable for solution or suspension in liquid prior to injection, or as emulsions. A more recently revised approach for parental administration involves use of a slow release or sustained release system, such that a constant level of dosage is maintained. See, e.g., U.S. Pat. No. 3,710,795, which is incorporated by reference herein. Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils (such as olive oil), and injectable organic esters (such as ethyl oleate). Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases, and the like.

For topical administration, if used, liquids, suspension, lotions, creams, gels, or the like may be used as long as the active compound can be delivered to the surface to be treated. Formulations for topical administration can also include ointments, drops, suppositories, sprays, and powders. Conventional pharmaceutical carriers, aqueous, powder or oily bases, thickeners, and the like can be necessary or desirable.

A cell can be in vitro. Alternatively, a cell can be in vivo and can be found in a subject. A “cell” can be a cell from any organism including, but not limited to, a human.

In one aspect, the compositions described herein can be administered to a subject, such as a human, that is in need of alleviation or amelioration from a recognized infective respiratory medical condition.

The dosages or amounts of the compositions described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex, and extent of the disease in the subject and can be determined by one of skill in the art. The dosage can be adjusted by the individual physician based on the clinical condition of the subject involved. The dose, schedule of doses, and route of administration can be varied.

The efficacy of administration of a particular dose of the compositions according to the methods described herein can be determined by evaluating the particular aspects of the medical history, signs, symptoms, and objective laboratory tests that are known to be useful in evaluating the status of a subject in need of treatment of respiratory infections, or other diseases and/or conditions. These. signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: 1) a subject's physical condition is shown to be improved (e.g., the respiratory symptoms such as increased mucous have lessened), 2) the progression of the disease or condition is shown to be stabilized, or slowed, or reversed, or 3) the need for other medications for treating the disease or condition is lessened or obviated, then a particular treatment regimen will be considered efficacious.

Methods

The invention includes a method of making the composition of the present invention. A method of making a terpene-containing composition that is effective for preventing and/or treating a respiratory infection comprises adding an effective amount of an effective terpene to a carrier solvent.

The terpenes and carriers are discussed above. The concentration at which each component is present is also discussed above. For example, 1000 ppm of citral can be added to water to form a true solution. As another example, 2000 ppm of citral can be added to water with a surfactant to form a stable suspension.

The method can further comprise adding a surfactant to the terepene-containing composition. Concentrations and types of surfactants are discussed above.

The method can further comprise mixing the terpene and carrier (e.g., water, saline, or buffer solution). The mixing is under sufficient shear until a “true” solution is formed. Mixing can be done via any of a number of high shear mixers or mixing methods. For example, adding terpene into a line containing water at a static mixer is expected to form a solution of the invention. With the more soluble terpenes, a true solution can be formed by agitating water and terpene by hand (e.g., in a flask). With lesser soluble terpenes, homogenizers, or blenders provide sufficient shear to form a true solution. With the least soluble terpenes, methods of adding very high shear are needed, or if enough shear cannot be created, can only be made into the desired mixture by addition of a surfactant.

Mixing the terpene and water with a solution-forming amount of shear instead of adding a surfactant will produce a true solution. A solution-forming amount of shear is that amount sufficient to create a true solution as evidenced by a final clear solution as opposed to a cloudy suspension or emulsion.

Citral is not normally miscible in water. Previously in the art, a surfactant has always been used to get such a terpene into solution in water. The present invention is able to form a solution of up to 1000 ppm in water by high shear mixing, and thus, overcome the necessity of a surfactant in all solutions.

Of the terpenes tested, citral has been found to form a solution at the highest concentration level in water.

In a large-scale production, the terpene can be added in line with the water and the high shear mixing can be accomplished by a static inline mixer.

Any type of high shear mixer will work. For example, a static mixer, hand mixer, blender, or homogenizer will work.

Infections in or on subjects are caused by a variety of organisms. For example, these organisms include bacteria, viruses, mycoplasmas, or fungi. The present invention is effective against any of these classifications of infective agents, in particular, bacteria, mycoplasmas, and fungi.

Examples of these infective agents are Staphylococcus aureus, Aspergillius fumigatus, Mycoplasma iowae, Sclerotinta homeocarpa, Rhizoctonia solani, Colletotrichum graminicola, Penicillum sp., and Mycoplasma pneumoniae

The compositions and methods of the present invention are effective in preventing or treating many, if not all, of these infections in a great variety of subjects, including humans and avians.

The invention includes a method of treating and/or preventing a respiratory infection. The method comprises administering a composition of the present invention to a subject.

The composition of this invention can be administered by a variety of means. For example, the composition can be administered by an aerosol nasal spray to humans.

The life span/breakdown time of the terpenes, as indicated above, should be taken into account when formulating a treatment schedule for prevention or treatment according to the present invention.

### ADDITIONAL REFERENCES

1. Boyanova, L. and G. Neshev, 1999. Inhibitory effect of rose oil products on Helicobacter pylori growth in vivo: preliminary report. J. Med. Microbiol. 48: 705-706.

2. Onawunmi, G. O., 1989. Evaluation of the antimicrobial activity of citral. Letters in Applied Microbiology 9(3): 105-108.

3. Wright, D. C., 1996. Antimicrobial oil-in-water emulsions. U.S. Pat. No. 5,547,677.

4. Wright, D. C., 1996. Antimicrobial oil-in-water emulsions. U.S. Pat. No. 5,549,901.

5. Wright, D. C., 1997. Antimicrobial oil-in-water emulsions. U.S. Pat. No. 5,618,840.

6. Wright, D. C., 1997. Micellar nanoparticles. U.S. Pat. No. 5,629,021.

7. Wright, D. C., 1997. Oil containing lipid vesicles with marine applications. U.S. Pat. No. 5,662,957.

8. Wright, D. C., 1997. Lipid vesicles having a bilayer containing a surfactant with anti-viral and spermicidal activity. U.S. Pat. No. 5,700,679.

9. Wright, D. C., 1998. Oral vaccine against gram negative bacterial infection. U.S. Pat. No. 5,730,989.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by volume, temperature is in ° C. or is at ambient temperature, and pressure is at or near atmospheric. There are numerous variations and combinations of the compositions and conditions for making or using them, e.g., component concentrations, desired solvents, solvent mixtures, temperatures, pressures, and other ranges and conditions that can be used to optimize the results obtained from the described compositions and methods. Only reasonable and routine experimentation will be required to optimize these.

### Example 1

### Preparation of the Terpene Mixture With Surfactant

The terpene, terpene mixture, or liposome-terpene(s) combination comprised a blend of generally recognized as safe (GRAS) terpenes with a GRAS surfactant. The volumetric ratio of terpenes was 1-99%, and the ratio of surfactant was 0-99% of the composition.

The terpenes, comprised of natural or synthetic terpenes, used were citral, b-ionone, eugenol, geraniol, carvone, terpeniol, or other terpenes with similar properties. The surfactant was Tween® 80 or other suitable GRAS surfactant. The terpenes were added to water.

### Example 2

### Preparation of a Terpene Solution Without Surfactant

Alternatively, the solution can be prepared without a surfactant by placing the terpene, e.g., citral, in water and mixing under solution forming shear conditions until the terpene is in solution.

The terpene-water solution was formulated without a surfactant. 100 ppm to 2000 ppm of natural or synthetic terpenes, such as citral, b-ionone, geraniol, carvone, terpeniol, or other terpenes with similar properties, were added to water and subjected to a high-shear blending action that forced the terpene(s) into a true solution. The terpene and water were blended in a household blender for 30 seconds. Alternatively, moderate agitation also prepared a solution of citral by shaking by hand for approximately 2-3 minutes.

The maximum level of terpene(s) that was solubilized varied with each terpene. Examples of these levels are as follows.

TABLE 1
Solution levels for various terpenes.

| Terpene | Level |
|---|---|
| Citral | 1000 ppm |
| Terpeniol | 500 ppm |
| b-ionone | 500 ppm |
| Geraniol | 500 ppm |
| Carvone | 500 ppm |

### Example 3

### Potency of Solution

Terpenes will break down in the presence of oxygen.

Citral, for example, is an aldehyde and will decay (oxygenate) over a period of days. A 500 ppm solution will lose half its potency in 2-3 weeks.

### Example 4

### In vitro Effectiveness of Terpenes Against Several Microorganisms

In vitro effectiveness of terpene compositions against various organisms was tested. The effectiveness of a terpene mixture solution comprising 10% by volume polysorbate 80, 10% b-ionone, 10% L-carvone, and 70% citral (lemon grass oil) against Escherichia coli, Salmonella typhimurium, Pasteurella mirabilis, Pseudomonas aeruginosa, Staphylococcus aureus, Candida albicans, and Aspergillius fumigatus was tested. The terpene mixture solution was prepared by adding terpenes to the surfactant. The terpene/surfactant was then added to water. The total volume was then stirred using a stir bar mixer.

Each organism, except A. fumigatus, was grown overnight at 35-37° C. in tryptose broth. A. fumigatus was grown for 48 hours. Each organism was adjusted to approximately 10⁵ organisms/ml with sterile saline. For the broth dilution test, terpene mixture was diluted in sterile tryptose broth to give the following dilutions: 1:500, 1:1000, 1:2000, 1:4000, 1:8000, 1:16000, 1:32000, 1:64000, and 1:128000. Each dilution was added to sterile tubes in 5 ml amounts. Three replicates of each series of dilutions were used for each test organism. One half ml of the test organism was added to each series and incubated at 35-37° C. for 18-24 hours. After incubation the tubes were observed for growth and plated onto blood agar. The tubes were incubated an additional 24 hours and observed again. The A. fumigatus test series was incubated for 72 hours. The minimum inhibitory concentration (MIC) for each test organism was determined as the highest dilution that completely inhibited the organism.

TABLE 2
Results of the inhibitory activity of different dilutions of terpene composition.

| | | | | Growth After Subculture | | | Mean |
|---|---|---|---|---|---|---|---|
| | Visual Assessment of Growth* | | | to Agar Plates* | | | Inhibitory |
| Organism | 1 | 2 | 3 | 1 | 2 | 3 | Dilution |
| S. typhimurium | 500 | 500 | 500 | 500 | 500 | 500 | 500 |
| E. coli | 1000 | 1000 | 1000 | 1000 | 1000 | 1000 | 1000 |
| P. mirabilis | 1000 | 1000 | 1000 | 1000 | 1000 | 1000 | 1000 |
| P. aureginosa | NI** | NI | NI | NI | NI | NI | NI |
| S. aureus | 1000 | 1000 | 1000 | 1000 | 1000 | 1000 | 1000 |
| C. albicans | 1000 | 1000 | 1000 | 1000 | 1000 | 1000 | 1000 |
| A. fumigatus | 8000 | 16000 | 16000 | 8000 | 16000 | 16000 | 13300 |

*The results of the triplicate test with each organism as the reciprocal of the dilution that showed inhibition/killing.
**NI = not inhibited.

### Example 5

### Effects of Terpene on Growth of Mycoplasma iowae

Effects of neat citral on growth of Mycoplasma iowae was studied. M. iowae is a known avian respiratory disease agent.

Three concentrations (500 ppm, 250 ppm, and 125 ppm) of citral in sterile DI water were prepared.

Mycoplasma iowae were incubated at 37° C. in R₂ (Chen, T. A., J. M. Wells, and C. H. Liao. 1982. Cultivation in vitro: spiroplasmas, plant mycoplasmas, and other fastidious, walled prokaryotes. pp. 417-446. in Phytopathogenic prokaryotes, V. 2, M. S. Mount and G. H. Lacy (ed.), Academic Press, New York) broth.

One to 2-day old cultures were observed under a dark-field microscope to ensure cells were in filamentous form before treatment. Cell suspensions were vortexed to ensure they were evenly mixed before and an aliquot of 0.5 mL was dispensed into a sterile tube.

One half of 1 mL of each terpene solution was added into each cell suspension tube. Thus, the final concentrations of citral were 250 ppm, 125 ppm, and 62.5 ppm, respectively. The cell suspension that was added with 0.5 mL of sterile water was used as a control.

The treated cell suspension was incubated for 24 hrs before the color changing units (CCUs) were determined by a 10-fold serial dilution in fresh R₂. All treatments were duplicated. The CCUs were determined to 10-8 for terpene concentrations of 250 ppm and 125 ppm, and to 10-9 for a terpene concentration of 62.5 ppm and sterile water.

All culture tubes were incubated for 15 days before final readings were taken.

TABLE 3
Results of citral in vitro against Mycoplasmas iowae.

| | Treatment | | | |
|---|---|---|---|---|
| | Water-treated | 62.5 ppm | 125 ppm | 250 ppm |
| Organism | (CCUs) | | | |
| M. iowae | 10⁹ | 10⁸ | 10⁸ | 10⁷ |

A comparison was made of the effect of 24-hr and 48-hr treatment times. The CCUs were determined by taking treated cell suspension from the same treated tube 24 hrs or 48 hrs after treatment.

TABLE 4
24 and 48 hour treatment comparisons.

| | Treatment (ppm) | | | | | | | |
|---|---|---|---|---|---|---|---|---|
| | Water- | Water- | | | | | | |
| | treated | treated | 62.5 | 62.5 | 125 | 125 | 250 | 250 |
| | 24 hr | 48 hr | 24 hr | 48 hr | 24 hr | 48 hr | 24 hr | 48 hr |
| Organism | (CCUs) | | | | | | | |
| M. iowae | 10⁷ | 10⁶ | 10⁶ | 10⁶ | 10⁷ | 10⁶ | 10⁵ | 10⁴ |

The results indicate that citral may be able to serve as a chemical for control of avian respiratory diseases when used at higher than 250 ppm and treated for a sufficient length of time.

### Example 6

### In vitro Effectiveness of Different Terpene Formulations Against Escherichia coli, Salmonella typhimurium, Pasteurella mirabilis, Pseudomonas aeruginosa, Staphylococcus aureus, Candida albicans, and Aspergillus fumigatus

This example shows the amount and types of terpenes from six different terpene formulations (Table 5) used for antimicrobial testing.

In the microbiological study, seven microorganisms including Escherichia coli, Salmonella typhimurium, Pasteurella mirabilis, Pseudomonas aeruginosa, Staphylococcus aureus, Candida albicans, and Aspergillus fumigatus were utilized. These microorganisms were selected in view that they are commonly present in infections and contaminate animal products utilized for human consumption. Each organism, except A. fumigatus, was grown overnight at 35-37° C. in tryptone broth. A. fumigatus was grown for 48 hours. Each organism was adjusted to approximately 10⁵ organisms/ml with sterile saline.

Each terpene formulation was diluted to 1:500, 1:1000, 1:2000, 1:4000, 1:8000, and 1:16000 in broth and/or saline.

Each terpene formulation dilution was added to sterile tubes in 5 ml amounts, and 5 ml of the test organism was added to each series and incubated for 1 hour. There were three replicates of each series of dilutions for each test organism.

After incubation, 0.5 ml of each tube was plated onto blood agar and incubated 18-24 hours at 35-37° C. The A. fumigatus test series was incubated for 72 hours at 25° C.

The minimum inhibitory concentration (MIC) for each test organism was determined as the highest dilution that completely inhibits the organism growth. The microbiological results are presented in Table 6.

TABLE 5
Terpene formulation used for antimicrobial testing.

Formulas (%)

| Terpene/Ingredient | A | B | C | D | E | F |
|---|---|---|---|---|---|---|
| Citral | | 15 | | | 20 | 70 |
| Carvone | | | 55 | 55 | 35 | 10 |
| Eugenol | | | 35 | | 40 | 10 |
| b-ionone | 30 | 80 | 10 | 40 | | |
| Liposome | 70 | | | | | |
| Tween ® 80 | | 5 | 5 | 5 | 5 | 10 |

TABLE 6
Effect of terpene formulations on microorganism growth.
DILUTION AT WHICH MICROORGANISM
GROWTH WAS INHIBITED

| | Formula | | | | | |
|---|---|---|---|---|---|---|
| Organism | A | B | C | D | E | F |
| E. coli | NI | NI | NI | NI | 2000 | 1000 |
| P. aeruginosa | NI | NI | NI | NI | 2000 | NI |
| P. mirabilis | NI | NI | NI | NI | 1000 | 1000 |
| S. typhimurium | NI | NI | NI | NI | 2000 | 500 |
| S. aureus | NI | 4000 | 1000 | 4000 | 2000 | 1000 |
| C. albicans | NI | 1000 | 2000 | 2000 | 2000 | 1000 |
| A. fumigatus | NI | NI | NI | NI | 500 | 13300 |

The results are expressed as the reciprocal of the dilution that showed biocidal effect.
NI = not inhibited

### Example 7

### In vitro Effectiveness of Terpenes Against Fungal Microorganisms: Sclerotinta homeocarpa, Rhizoctonia solani, and Colletotrichum graminicola

Two terpene formulations were tested against Sclerotinta homeocarpa, Rhizoctonia solani, and Colletotrichum graminicola. Formula A contained 40% eugenol, 35% 1-carvone, 20% citral, and 5% Tween® 80. Formula B contained 70% citral, 10% b-ionone, 10% 1-carvone, and 10% Tween® 80.

Potato dextrose agar media was amended with each terpene formulation to make a 5000 ppm final concentration of each.

For each pathogen, a 5 mm diameter agar plug containing fungal micelia was transferred to each of 5 plates for both terpene formulation and control. All plates were parafilmed and incubated at 25° C. The diameter of fungal colony growth was measured (mm) and recorded. When the control plates were full, measurements were stopped. Colony area was calculated using π r², where r is the radius of the colony.

TABLE 7
Effect of terpenes on fungal growth (area = mm²).

| | S. homeocarpa | | R. solani | | C. graminicola | |
|---|---|---|---|---|---|---|
| Treatment | Day 1 | Day 2 | Day 1 | Day 2 | Day 2 | Day 7 |
| Formula A | 0 | 0 | 0 | 0 | 0 | 0 |
| Formula B | 0 | 0 | 0 | 0 | 0 | 0 |
| Control | 209.0 | 2023.2 | 162.3 | 1976.6 | 136.7 | 2023.2 |

### Example 8

### In vivo Effectiveness of Single or Combination of Terpenes Against E. coli

The objective of this example was to determine a terpene mixture that could have an optimal effect.

E. coli strain AW574 was grown in tryptone broth to an exponential growth phase (O.D. between 0.4 and 1.0 at 590 nm). One tenth of this growth was inoculated to 10 ml of tryptone broth followed by the addition of individual terpenes or as indicated on Table 6; then incubated for 24 hours at 35-37° C., and the O.D. determined in each tube. The concentration of terpenes was 1 or 2 μMol. Each treatment was repeated in triplicate. The results are expressed as percentage bacterial growth as compared to the control treatment.

It is observed that the combination of terpenes gives better biocidal effect than single terpenes, with geraniol and carvone appearing to be better than b-ionone.

TABLE 8
Effect of single terpene or their combination
against E. coli growth.

| μMol terpenes | | | % |
|---|---|---|---|
| b-ionone | Carvone | Geraniol | growth |
| 0 | 0 | 0 | 100.00 |
| 2 | 0 | 0 | 84.00 |
| 0 | 2 | 0 | 63.00 |
| 0 | 0 | 2 | 54.00 |
| 1 | 1 | 1 | 41.00 |
| 1 | 2 | 1 | 31.10 |
| 1 | 1 | 2 | 14.80 |
| 1 | 2 | 2 | 15.90 |
| 2 | 1 | 1 | 48.60 |
| 2 | 2 | 1 | 44.30 |
| 2 | 1 | 2 | 30.20 |
| 2 | 2 | 2 | 1.50 |

### Example 9

### Nasal Spray

This example shows a bioactive terpene formulation containing 50% v/v carvone, 30% eugenol, 10% eucalyptus oil, and 10% Tween® 80.

The solution was prepared by mixing the terpenes first and then adding Tween® 80. This mixture was diluted in a standard 0.9% saline. After the solution was agitated, it was stored in an off-the-shelf nasal sprayer.

In this formulation, eugenol was acting as antimicrobial and anesthetic; the eucalyptus oil dilates nasal passages; and carvone is also an antimicrobial. This formulation was effective against bacteria and fungi that may be present in the respiratory system.

A preliminary study showed that at 2000 ppm the formulation produced slight irritation. Reducing the concentration to 1000 ppm eliminated this problem.

Experimental Conditions

Formulation 1

20% v/v citral, 50% b-ionone, 20% 1-carvone, and 10% Tween® 80

Formulation 2

50% v/v 1-carvone, 10% b-ionone, 20% eugenol, and 10% Tween® 80

Formulation 3

50% v/v 1-carvone, 30% eugenol, 10% purified eucalyptus oil, and 10% Tween® 80

All formulations were prepared by first mixing the terpenes and then adding the Tween® 80. The Tween® 80 is used only as an emulsifier. The terpenes were then diluted in a standard 0.9% saline solution in two batches: 1000 ppm and 2000 ppm active terpene. The three formulations in two concentrations were transferred to standard off-the-shelf nasal sprayers.

Six adults were given six sprayers each and asked to try them in random order over a period of three days noting their reactions.

Results

Formulation 1 produced a burning sensation at both 1000 and 2000 ppm. This may be due to the citral.

Formulation 2 produced a similar irritation at both levels, but the 1000 ppm level stopped burning after one minute.

Formulation 3 produced slight burning at 2000 ppm, but produced no irritation at 1000 ppm. This formulation had the advantage of opening the sinus passages on two subjects who had sinusitis.

Conclusion

Formulation 3 at 1000 ppm was judged the most suitable for controlling bacteria and fungi in the nasal passages and lungs.

### Example 10

### Red Blood Cell Lysing Study

Protocol:

5 ml of PBS was added to terpene (100, 250, 500, 1000, and 2000 ppm). 0.050 ml of heparinized blood was then added to these mixtures. These mixtures were incubated for 20 minutes.

The samples were then centrifuged at 2000 rpm for 5 min and read at 540 nm.

Lysing of red cells in the terpene mixtures was compared to control, or to 0 ppm terpene.

Study 1: Chicken Blood and B-Ionone

TABLE 9
Destruction of red blood cells with b-ionone vs. control.

O.D.

| Concentration | | B-ionone |
|---|---|---|
| (ppm) | B-ionone + 10% Tween ® 80 | neat |
| 0 | 0.012 | 0.012 |
| 100 | 0.029 | 0.013 |
| 500 | 0.345 | 0.056 |
| 1000 | 0.576 | 0.038 |
| 2000 | 0.944 | 0.106 |

Lysing of the red cells occurred with terpene concentration of 500 ppm without Tween® and 100 ppm with Tween® 80.

Study 2: Human Blood, b-Ionone, Citral, and 1-Carvone

TABLE 10
Concentration at which erythrocytes lyse
in the presence of various terpenes.

| | Concentration at which erythrocytes lyse (ppm) | |
|---|---|---|
| Terpene | Neat | +10% Tween ® 80 |
| b-ionone | 2000 | 2000 |
| Citral | 1000 | 500 |
| 1-Carvone | 250 | 250 |

This demonstrates the levels at which undesirable side effects can occur from terpenes if they enter the blood stream, for example, by passing through the nasal mucosa.

### Example 11

### Summary of Mold Studies

TABLE 11
Formulas tested.

| Terpene (%) | A | FW | B | C | D | E | F |
|---|---|---|---|---|---|---|---|
| Citral | 20 | 70 | 10 | 30 | 60 | 100 | 95 |
| 1-Carvone | 35 | 10 | 50 | 30 | 30 | — | — |
| Eugenol | 40 | — | 30 | 30 | — | — | — |
| b-ionone | — | 10 | — | — | — | — | — |
| Tween ® 80 | 5 | 10 | 10 | 10 | 10 | — | 5 |

Study 1:

1. Mold spores, Penicillum sp., were mixed with 1000 ppm of terpene formulation as indicated in the table and added to a Potato-Dextrose agar plate.

2. After 48 h incubation the plates showed the following results:

F<E<C<FW<D<A<B<Control.

3. After 72 hours the plates showed the following results:

F<E<C<FW<D<A<B<Control.

Formulas F and E performed better than the others.

Study 2:

1. Mold spores, Penicillum sp., were mixed with 1000 ppm of each terpene formulation, incubated for 1 hour, and then added to Potato-Dextrose agar plates.

2. After 48 h incubation, the plates showed the following results:

F<FW<E<D<C<B<A<Control.

Formulas F and E performed better than the others.

Study 3:

1. Mold spores, Penicillum sp., were mixed with 1000 ppm of each terpene formulation, incubated for 24 hours, and then added to Potato-Dextrose agar plates.

2. After 48 h incubation, the plates showed the following results:

F<E<D<FW<C<A<B<Control

Formulas F and E performed better than the others.

Test were repeated several times with the same results. Formulas E and F performed better that the others.

### Example 12

### Biofilm Formation and Testing

### Destruction of Biofilm

In 96-well polystyrene plates or PVC plates

1. Add 100 ml of bacterial culture in nutrient broth, culture has to be made fresh by adding 1-2 ml of 1×10⁶ cfu in 50-100 ml broth and incubating overnight (14-18 h) at 37° C.

2. Incubate overnight at 35-37° C. This will develop a biofilm.

3. Wash 4 times with water.

4. Add 100 ml of 1:1000 terpene solution.

5. Let incubate for 1 hour or more depending on test protocol.

6. Add 25 μl of 1% crystal violet. This is done to quantify the biofilm formation. Dye will coat bacteria attached to wells.

7. Incubate for 15 minutes.

8. Wash wells four times with water and blot dry.

9. Add 200 μl 95% ethanol, mix.

10. In a new plate, transfer 150 μl solution to clean wells.

11. Read at 590 nm.

12. Results are expressed as the difference between OD of control as compared to treated samples.

Study 1:

Four terpene formulations with two type of surfactants, a total of eight formulas (A, B, C and D with 10% Tween® 80, H, J, K and L have 10% Span® 20) were prepared. Formulas A-D are those used in Example 11 with 10% Tween® 80. H-L are Formulas A-D from Example with 10% Span® 20.

TABLE 12
Formulas tested vs control for reduction in biofilm achieved.

| Formula | OD test | OD control | % reduction |
|---|---|---|---|
| A | 0.098 | 0.210 | 53 |
| B | 0.187 | 0.220 | 15 |
| C | 0.220 | 0.229 | 4 |
| D | 0.295 | 0.230 | 0 |
| H | 0.223 | 0.230 | 3 |
| J | 0.273 | 0.194 | 0 |
| K | 0.233 | 0.194 | 0 |
| L | 0.153 | 0.194 | 0 |

Study 2:

Destruction of Biofilm by Terpenes

Five formulas with their results. The formulas correspond to those used in Example 11.

TABLE 13
Formulas tested vs control for reduction in biofilm achieved.

| Formula | OD test | OD control | % reduction |
|---|---|---|---|
| A | 0.299 | 0.459 | 35 |
| FW | 0.437 | 0.459 | 5 |
| B | 0.284 | 0.459 | 38 |
| C | 0.264 | 0.459 | 42 |
| D | 0.247 | 0.459 | 46 |

### Example 13

### Biofilm Formation and Testing

### Prevention of Biofilm Formation

In 96-well polystyrene plates or PVC plates

1. Add 50 ml of bacterial culture in nutrient broth, culture has to be made fresh by adding 1-2 ml of 1×10⁶ cfu in 50-100 ml broth and incubating overnight (14-18 h) at 37° C.

2. Add 100 ml of 1:1000 terpene solution.

3. Incubate overnight at 35-37° C. This will develop a biofilm.

4. Wash 4 times with water.

5. Add 25 μl of 1% crystal violet. This is done to quantify the biofilm formation. Dye will coat bacteria attached to wells.

6. Incubate for 15 minutes.

7. Wash wells four times with water and blot dry.

8. Add 200 μl 95% ethanol, mix.

9. In a new plate, transfer 150 μl solution to clean wells.

10. Read at 590 nm.

11. Results are expressed as the difference between OD of control as compared to treated samples.

### Example 14

### Determination of Citral in Water Samples

Reagents: Schiff reagent is diluted 1:10 with distilled water.

1. In test tubes, add 1 ml of solution to be tested.

2. Add 0.1 ml of 1:10 Schiffreagent.

3. Incubate at room temperature for 10 minutes.

4. Reaction will turn from pink to blue, pink color is 0 ppm citral, reaction starts to turn blue above 100 ppm.

### Example 15

### In vitro Effectiveness of Terpenes Against Mycoplasma pneumoniae

Terpene beta-ionone or L-carvone was first mixed well with Tween® 80 to have a final Tween® 80 concentration of 5%. This mixture was then used to make concentrations of 2500 ppm in sterile phosphate buffer saline (PBS) by blending the mixture in PBS for 40 seconds. This 2500 ppm solution was then diluted to 500 ppm, 250 ppm, and 125 ppm with PBS.

PBS containing 25 ppm Tween® 80 or PBS alone was used to treat cells suspension as controls.

A log phase (2-3-day old) culture of Mycoplasma pneumoniae was mixed with each of the above three concentrations of terpene at 1:1 (volume) ratio (in this case, 1 mL of cell suspension was added to 1 mL of terpene).

The culture and terpene mixture was then incubated at 37° C. for 40 hours. After 40 hours of treatment, 10-fold serial dilution was performed to 10 (−10) by first taking 0.1 mL of the treated culture suspension was added into 0.9 mL of fresh SP4 (Whitcomb (1983); SP4 media is commercially available (Remel, Lenexa, Kans., USA)). All the tubes were then incubated at 37° C., and a color change of the medium was used for the indication of the cells that either were killed or survived from the treatment. Color change was from red to yellow because Mycoplasma pneumoniae produces acid during its growth.

Three days after the 10-fold dilution, the first tube of the following treatments has changed color from red to yellow indication no killing effects: PBS, PBS containing 25 ppm Tween® 80, 62.5 ppm L-carvone, 125 ppm L-carvone, and 250 ppm L-carvone, whereas those treated with 62.5 ppm, 125 ppm, and 250 ppm of beta-ionone did not change color at all indicating a killing effect of ionone on Mycoplasma pneumoniae. However, 6 days after the 10-fold dilution, the second and third tube of the PBS, PBS containing 25 ppm Tween® 80, 62.5 ppm L-carvone, 125 ppm L-carvone, and 250 ppm L-carvone changed color, whereas only the first tube of 62.5 ppm beta-ionone changed color indicating that beta-ionone at 125 and 250 ppm may have completely killed all cells in 40 hours.

All the treatments were performed in duplicate.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition for treating and/or preventing a respiratory infection in a subject comprising an effective amount of at least one effective terpene.

2. The composition of claim 1 wherein the composition is a solution.

3. The composition of claim 1 further comprising water.

4. The composition of claim 1 further comprising a surfactant and water.

5. The composition of claim 4 wherein the surfactant is polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, Tween®, Span® 20, Span® 40, Span® 60, Span® 80, or mixtures thereof.

6. The composition of claim 1 further comprising saline or a buffer solution.

7. The composition of claim 1 wherein the at least one terpene is a mixture of different terpenes.

8. The composition of claim 1 wherein the at least one terpene is a terpene-liposome combination.

9. The composition of claim 1 wherein the terpene comprises citral, pinene, nerol, b-ionone, geraniol, carvacrol, eugenol, carvone, terpeniol, anethole, camphor, menthol, limonene, nerolidol, farnesol, phytol, carotene (vitamin Al), squalene, thymol, tocotrienol, perillyl alcohol, borneol, myrcene, simene, carene, terpenene, linalool, or mixtures thereof.

10. The composition of claim 1 wherein the terpene is citral, carvone, b-ionone, eugenol, eucalyptus oil, or mixtures thereof.

11. The composition of claim 1 wherein composition comprises about 1 to 99% by volume terpenes and about 1 to 99% by volume surfactant.

12. The composition of claim 1 wherein the terpene comprises between about 100 ppm and about 2000 ppm.

13. The composition of claim 1 wherein the terpene comprises about 100 ppm.

14. The composition of claim 1 wherein the terpene comprises about 250 ppm.

15. The composition of claim 1 wherein the terpene comprises about 500 ppm.

16. The composition of claim 1 wherein the terpene comprises about 1000 ppm.

17. The composition of claim 1 wherein the terpene is 50% L-carvone, 30% eugenol, 10% purified eucalyptus oil and the effective amount is 1000 ppm, and wherein 10% is a surfactant.

18. The composition of claim 1 wherein the terpene is citral and the effective amount is 1000 ppm.

19. The composition of claim 18 wherein the composition further comprises 5% surfactant.

20. The composition of claim 1 wherein the terpene is b-ionone and the effective amount is 250 ppm and wherein 5% is a surfactant.

21. The composition of claim 1 wherein the terpene is effective against bacteria, mycoplasmas, and/or fungi.

22. The composition of claim 1 wherein the terpene is effective against bacteria.

23. The composition of claim 1 wherein the terpene is effective against mycoplasmas.

24. The composition of claim 1 wherein the subject is a human.

25. The composition of claim 1 wherein the subject is avian.

26. A composition for treating and/or preventing a respiratory infection in a subject comprising a solution comprising an effective amount of at least one effective terpene and water.

27. A pharmaceutical composition for treatment and/or prevention of a respiratory infection in a subject comprising an effective amount of an effective terpene and a pharmaceutically acceptable carrier.

28. The pharmaceutical composition of claim 27 wherein the composition is an aerosol solution.

29. A method for preventing and/or treating respiratory infection comprising administering a composition comprising an effective amount of an effective terpene to a subject.

30. The method of claim 29 wherein the composition further comprises water.

31. The method of claim 29 wherein the composition further comprises a surfactant.

32. The method of claim 29 wherein the administration is by spraying the respiratory tract of the subject with the composition.

33. The method of claim 32 wherein the spraying the composition is into the nasal cavity of the subject.

34. The method of claim 29 further comprising making a composition comprising an effective amount of an effective terpene.

35. The method of claim 29 wherein the subject is human.

36. The method of claim 29 wherein the subject is infected with an infective agent.

37. The method of claim 36 wherein the infective agent is bacteria, mycoplasmas, and/or fungi.

38. The method of claim 37 wherein the infective agent is bacteria.

39. The method of claim 37 wherein infective agent is mycoplasma.

40. The method of claim 34 wherein the making a composition comprises mixing an effective amount of an effective terpene and water.

41. The method of claim 40 wherein the mixing is done at a solution-forming shear until formation of a true solution of the terpene and water.

42. The method of claim 41 wherein the terpene mixed is into a true solution in water without a surfactant by high shear or high pressure blending or agitation.

43. The method of claim 42 wherein the solution-forming shear mixing is via a static mixer.

44. A method for preventing and/or treating a respiratory infection comprising administering a composition comprising an effective amount of an effective terpene and water to a subject.

45. The method of claim 44 wherein the composition is a true solution.

46. A method for making a terpene-containing composition effective for preventing and/or treating a respiratory infection comprising mixing a composition comprising a terpene and water at a solution-forming shear until a true solution of the terpene is formed.

47. A method for making the composition of claim 1 comprising mixing a terpene with a carrier.

48. A method for using the composition of claim 1 comprising administering the composition of claim 1 to an infected subject.
